(19) United States
(12) Patent Application Publication
(10) Pub. No.: US 2019/0328287 A1
(43) Pub. Date: Oct. 31, 2019

(54) ENTRY-EXIT INTERVENTION SYSTEM, METHOD, AND COMPUTER-READABLE MEDIUM

(71) Applicant: Konica Minolta Business Solutions U.S.A., Inc., Ramsey, NJ (US)

(72) Inventor: Yalan Lai, San Mateo, CA (US)

(21) Appl. No.: 15/965,784

(22) Filed: Apr. 27, 2018

(51) Int. Cl. A61B 5/1171; A61B 5/00; G08B 21/04; G06K 9/00

(52) U.S. Cl. CPC A61B 5/1176; A61B 5/746; A61B 2562/08; G06K 9/00221; G08B 21/0476

(57) ABSTRACT

The incidence of elopement of an at-risk person from a caregiving facility or other environment can be reduced by providing staff more time to intervene. This may be accomplished by a system having one more recording players positioned near an exit. The players serve to distract the at-risk person who may be wandering towards a controlled area, such as an exit door. The at-risk person may have dementia, and a recording player may play a song to which the at-risk person is known to have an emotional response. An identification sensor near the exit can be used to ascertain the identity of the at-risk person. A computer matches the ascertained identity to a recording of the song and instructs the player to play that song.

### FIELD

This disclosure relates generally to communications and, more particularly, communications for controlling entry/exit of persons to/from a space.

### BACKGROUND

Elopement refers to a situation when a patient or resident who is cognitively, physically, mentally, emotionally and/or chemically impaired wanders away, walks away, or otherwise leaves a caregiving facility or environment unsupervised, unnoticed and/or prior to their scheduled discharge. Security doors with locks and/or door alarms that sound when the door is opened have been used to reduce the incidence of elopement. However, a patient or resident may still be able to pass through a normally locked security door if he/she happens to follow unnoticed behind an authorized person or if the door is temporarily propped open. In addition, elopement may still occur even when there is a door alarm if staff is unable to intervene in a timely manner after the door alarm is activated. Further, a locked security door may not desired in some spaces due to fire safety concerns or may not be desired in an area of high foot traffic in a low-security environment. So as not to impede travel, a closed-circuit TV camera might be used to monitor the low security environment. Even if a patient or resident is detected on closed-circuit TV, elopement may occur if staff is unable to intervene in a timely manner.

Accordingly, there is a need for an elopement prevention scheme that gives staff more time to intervene.

### SUMMARY

Briefly and in general terms, the present invention is directed to a system, method, and non-transitory computer readable medium for entry-exit intervention.

In aspects of the invention, a system comprises an identification sensor configured to output sensor data for identifying various persons, a data store containing a plurality of recordings, each recording associated with one or more persons, a first recording player comprising one or both of a speaker and a display screen. The system further comprises an alarm, and a computer. The computer receives the sensor data, ascertains an identity of a person according to the received sensor data, determines whether the ascertained identity matches any of the recordings. When the ascertained identity matches any of the recordings, the computer activates the alarm and instructs the first recording player to play a first recording among the plurality of recordings that matches the ascertained identity.

In aspects of the invention, a method comprises ascertaining an identity of a person according to sensor data, determining that the ascertained identity matches any of a plurality of recordings contained in a data store, and upon such determination, activating an alarm and causing a first recording player to play a first recording among the plurality of recordings that matches the ascertained identity.

In aspects of the invention, a non-transitory computer readable medium has stored thereon computer readable instructions that, when executed by a computer, cause the computer to perform a method for an entry-exit intervention, which comprises ascertaining an identity of a person according to sensor data, determining that the ascertained identity matches any of a plurality of recordings contained in a data store, and upon such determination, activating an alarm and causing a first recording player to play a first recording among the plurality of recordings that matches the ascertained identity.

The features and advantages of the invention will be more readily understood from the following detailed description which should be read in conjunction with the accompanying drawings.

### BRIEF DESCRIPTION OF THE DRAWINGS

FIG. 1 is a schematic block diagram showing an example system for entry-exit intervention.

FIGS. 2-4 are diagrams showing example setups of the system in a controlled area.

FIG. 5 is a flow diagram showing an example method for entry-exit intervention.

### DETAILED DESCRIPTION

As used herein, the term “at-risk person” refers to a person who is at risk of elopement. That is, it is desired to prevent an at-risk person from passing through a controlled area unsupervised, unnoticed and/or prior to scheduled discharge. While the descriptions below refer to a caregiving facility, the present invention is not limited to such facilities. The present invention may be applied to other environments, including without limitation daycare centers for young children who would be at-risk persons in the present disclosure.

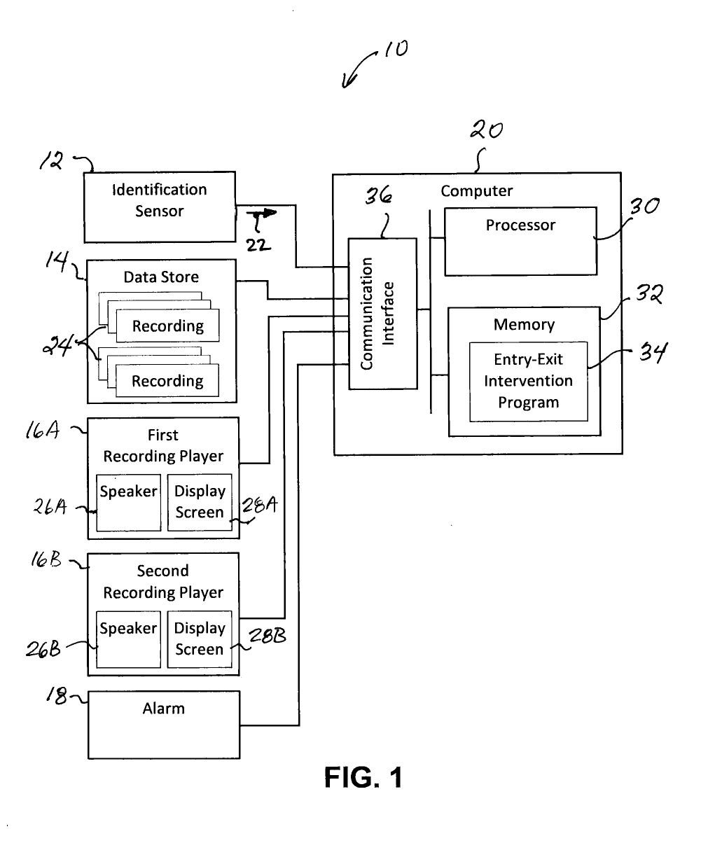

Referring now in more detail to the drawings for purposes of illustrating non-limiting examples, wherein like reference numerals designate corresponding or like elements among the several views, there is shown in FIG. 1 example system 10 for entry-exit intervention. As described below, system 10 may be used to distract an at-risk person during his/her course of travel, such as in a controlled area of a caregiving facility. Distraction may slow the person's travel through a door or any pathway, thus giving staff in the caregiving facility more time to intervene to prevent elopement.

System 10 comprises identification sensor 12, data store 14, first recording player 16A, alarm 18, and computer 20. Identification sensor 12 is configured to output sensor data 22 for identifying various persons. Identification sensor 12 may comprise an RFID reader configured to detect an RFID tag on a wearable item, such as wristband, on an at-risk person. The RFID tag encodes an identification code that is uniquely associated with that person. Additionally or alternatively, identification sensor 12 comprises a camera with a CCD, CMOS, or other type of image sensor. As will be discussed below, the camera can be used for facial recognition to identify an at-risk person.

Data store 14 contains a plurality of recordings 24, each of which are associated with one or more at-risk persons. For example and without limitation, recordings 24 may include the favorite song of an at-risk person. Other examples are discussed below. Data store 14 comprises non-volatile memory that stores recordings 24. Examples of non-volatile memory include without limitation a flash memory device, a magnetic storage device, and an optical disc.

First recording player 16A comprises audio speaker 26A and visual display screen 28A. In other aspects, first recording player 16A has only one of speaker 26A and display screen 28A. Examples for display screen 28A include without limitation LCD and OLED screens. Display screen 28A may include a touch-sensitive layer configured to sense touching by a person's finger. First recording player 16A is configured to play recordings 24 from data store 14. As used herein, the word “play” encompasses displaying a still image, such as a photograph, on display screen 28A without any audio output. First recording player 16A can be portable. Alternatively, first recording player 16A is not portable and can be located at a fixed location. For example, first recording player 16A can be mounted on a wall or fixed to the ground. Examples for first recording player 16A include without limitation a multi-media kiosk, a tablet computer, and a smartphone. The smartphone can be one that is owned by, assigned to, or otherwise uniquely associated with an at-risk person.

In some aspects, data store 14 may be a part of first recording player 16A. Alternatively, data store 14 is separate and distinct from first recording player 16A.

Alarm 18 is configured to alert staff to intervene to prevent elopement. Alarm 18 can be portable so a staff member can carry it. Alarm 18 is not portable and can be at a fixed location, such as a nurse station. Alarm 18 can be located remotely from first recording player 16A. For example, alarm 18 may not be visible or audible to an at-risk person located at first recording player 16A. In this way, alarm 18 will not cause an at-risk person to become distressed or run through a controlled area. Examples for alarm 18 include without limitation a mobile phone, a telephone, a computer, a bell, a buzzer, a light, and any combination thereof.

Computer 20 comprises processor 30, memory 32 that stores entry-exit intervention program 34, and communication interface 36. Computer 20 may comprise a keyboard, touch screen, or other means to allow a user to enter data. Processor 30 comprises circuits and electronic components that execute instructions of an operating system and entry-exit intervention program 34. Entry-exit intervention program 34 enables computer 20 to perform various processes and functions described herein. Example elements for memory 32 include without limitation random-access memory (RAM) modules, read-only memory (ROM) modules, and other electronic data storage devices. Memory 32 may include a mass storage type of device such as a solid-state flash drive, CD drive, and DVD drive. Memory 32 comprises a non-transitory computer readable medium that stores entry-exit intervention program 34 that contain instructions for performing various processes and functions described herein. Communication interface 36 comprises circuits and electronic components configured to send and receive data to/from identification sensor 12, data store 14, first recording player 16A, and alarm 18. Data communication may be achieved through electrical or optical cables. Data communication may be wireless, such as by using Wi-Fi technology. For example, computer 20 may be operatively coupled to identification sensor 12, data store 14, first recording player 16A, and alarm 18 by a Wi-Fi network.

In some aspects, data store 14 may be a part of computer 20. Alternatively, data store 14 is separate and distinct from computer 20.

Computer 20 is configured to receive sensor data 22 from identification sensor 12. Computer 20 is configured to ascertain an identity of a person according to the received sensor data. Computer 20 is configured to determine whether the ascertained identity matches any of the recordings. When the ascertained identity matches any of recordings 24, the person is deemed to be an at-risk person, so computer 20 activates alarm 18 and instructs first recording player 16A to play a first recording among the plurality of recordings 24. The first recording is one of recordings 24 that matches the ascertained identity.

For example, computer 20 may store a list of identification codes that are uniquely associated with various at-risk persons, and sensor data 22 may include an identification code obtained from an RFID tag on a particular at-risk person. The identification code is obtained from the RFID tag by an RFID reader of identification sensor 12. Ascertaining the identity of the person may comprise matching the obtained identification code to one of the identification codes in the stored list of at-risk persons.

In some cases, an at-risk person may not have an RFID tag. For example, the person might have removed a wristband that carries the RFID tag, or the RFID tag may become inadvertently separated from the person. To address this issue, identification sensor 12 may comprise a camera configured to output an image of the person as sensor data 22. The camera may be in addition to or an alternative to an RFID reader. Computer 20 runs a facial recognition algorithm on the image (sensor data 22) from the camera to ascertain the identity of the person. For example, computer 20 may store data sets of facial characteristics that are uniquely associated with various at-risk persons. Computer 20, by means of the facial recognition algorithm, determines facial characteristics of the person in the image and matches the determined facial characteristics to one of the sets of facial characteristics.

First recording player 16A begins to play the first recording when instructed by computer 20. As indicated above, the first recording is associated with the at-risk person who was identified by computer 20. The first recording is personalized to that person in the sense that first recording player 16A may play a different recording if another at-risk person had been identified by computer 20. The first recording can be any one or any combination of a music recording, a song recording, and a video recording. For example, first recording can be a song, a photograph, a recording of football game or other sporting event, a recording of a cinematic move or TV show, or an interactive video game.

The first recording is intended to attract the attention of the identified at-risk person, thereby distracting him or her from passing through a controlled area, such as a door, hallway, lobby, or other space. Persons with dementia are particularly responsive to certain songs and music from their past. This may be due to a strong emotional connection to certain songs and music. For example, a song may be known by staff to elicit a positive emotional response in an at-risk person, so a recording of the song is stored in data store 14 in association with the at-risk person. Later, when a person approaches the controlled area and is identified by computer 20 to be the at-risk person, the recording of the song (an example of the first recording) will be played by first recording player 16A. The song delays the person's travel through the controlled area. In the meantime, alarm 18 summons another person (such as a staff member, nurse, or guardian) to the at-risk person while the at-risk person's travel is delayed.

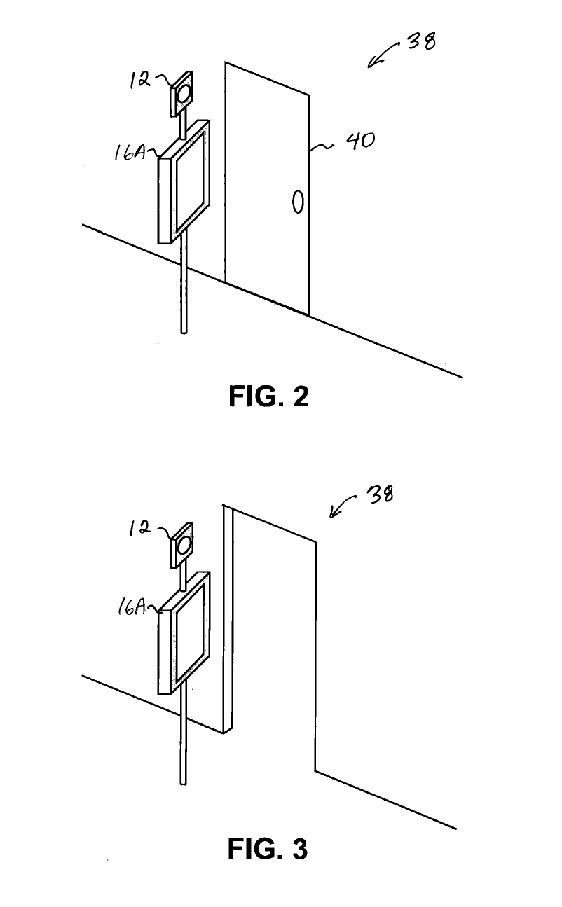

FIG. 2 shows example controlled area 38 in which system 10 has been setup. Controlled area 38 has door 40 as part of system 10. For example, first recording player 16A may be located on the interior side of door 40. Thus, an at-risk person's travel may be delayed by system 10 before the person passes through door 40, thereby giving more time for a staff member to intervene and guide the person away from door 40. Alternatively, first recording player 16A may be located on the exterior side of door 40. In that case, an at-risk person's travel may be delayed by system 10 after exiting door 40, thereby giving more time for a staff member to intervene and guide the person back inside.

Door 40 may be a locked security door, and system 10 may provide an extra measure of security to prevent elopement in case an at-risk person follows an authorized person out door 40. Alternatively, door 40 may not be locked because controlled area 38 may be a low security area with high foot traffic. Thus, door 40 is not locked when first recording player 16A plays the first recording. If an at-risk person approaches door 40 or passes through the unlocked door, that person's travel may be delayed by system 10 to allow more time for a staff member to intervene.

FIG. 3 shows another example controlled area 38 in which system 10 has been setup. Controlled area 38 has no door. Controlled area 38 can be a hallway or other open space. If an at-risk person approaches or travels across controlled area 38, that person's travel may be delayed by system 10 to allow more time for a staff member to intervene to prevent elopement.

Referring again to FIG. 1, system 10 may comprise second recording player 16B, that comprises audio speaker 26B and visual display screen 28B. In other aspects, second recording player 16B has only one of speaker 26B and display screen 28B. Descriptions for first recording player 16A apply to second recording player 16B. Descriptions for display screen 28A apply to display screen 28B.

Second recording player 16B is positioned so that it guides an at-risk person further away from a door or other controlled area. For example, second recording player 16B can be fixed at a location that is least 2 meters from first recording player 16A, which is closer to a door or other controlled area. When the ascertained identity of a person matches any of recordings 24, the person is deemed to be an at-risk person and the computer instructs the second recording player to play the first recording which is associated with the at-risk person.

In order to guide the at-risk person away from the controlled area, first recording player 16A and second recording player 16B play the first recording with staggered timing. In a non-limiting example of staggered timing, first recording player 16A and second recording player 16B may begin playing the first recording at the same time. After a delay time period, first recording player 16A fades the first recording or stops playing the first recording. Fading comprises any of decreasing sound volume and darkening display screen 28A. The delay time period can be from 10 to 60 seconds, or from 10 to 30 seconds. Meanwhile, second recording player 16B continues to play the first recording. After the delay time period, second recording player 16B optionally boosts the first recording. Boosting comprises increasing sound volume and brightening display screen 28B.

In another example of staggered timing, second recording player 16B starts to play the first recording after first recording player 16A has started to play the first recording. That is, first recording player 16A starts to play the first recording, then after a delay time period, second recording player 16B starts to play the first recording. The delay time period can be from 10 to 60 seconds, or from 10 to 30 seconds.

Instead of playing the first recording, second recording player 16B may play a second recording among the plurality of recordings that matches the ascertained identity. For example, the first recording may be a first song known by the at-risk person who has been identified by computer 20. The second recording may be a second song known by the at-risk person.

In the descriptions above, second recording player 16B plays a recording with staggered timing relative to first recording player 16A. There can be a series of more than two recording players to guide the at-risk person even further away from a controlled area. As described below, each subsequent recording player in the series plays a recording with staggered timing relative to the previous recording player.

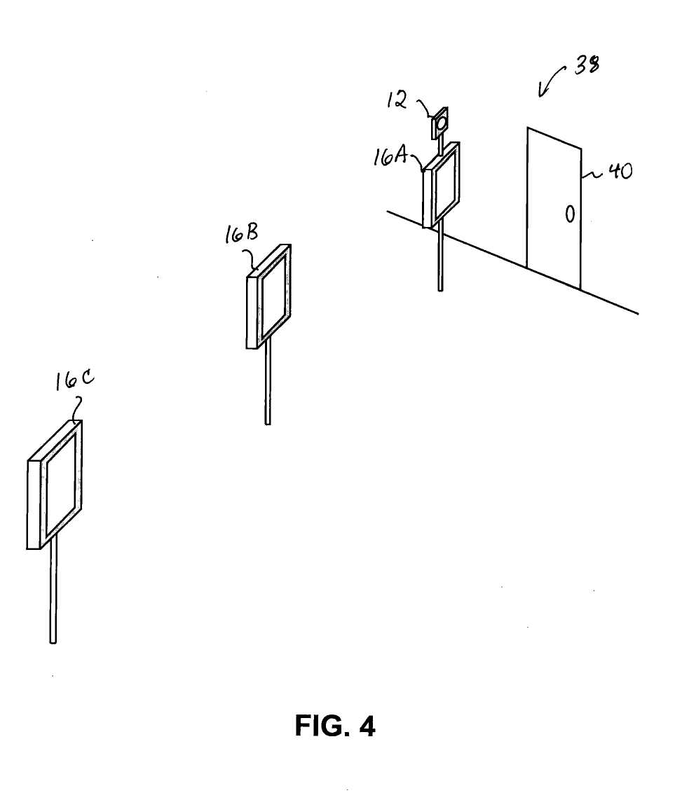

In FIG. 4, system 10 comprises third recording player 16C. Third recording player 16C may be 2 to 20 meters from second recording player 16B. Descriptions for first recording player 16A apply to third recording player 16C. In a non-limiting example of staggered timing, first recording player 16A, second recording player 16B, and third recording player 16C may begin playing the first recording at the same time. After a delay time period, first recording player 16A fades the first recording or stops playing the first recording. Meanwhile, second recording player 16B and third recording player 16C continue to play the first recording and optionally boost the first recording. After another delay time period, second recording player 16B fades the first recording or stops playing the first recording. Meanwhile, third recording player 16C continues to play the first recording and optionally boosts the first recording.

In another example of staggered timing, first recording player 16A starts to play the first recording, then after a delay time period, second recording player 16B starts to play the first recording. Then after another delay time period, third recording player 16C starts to play the first recording.

Instead of playing the first recording, third recording player 16B may play a third recording among the plurality of recordings that matches the ascertained identity. For example, first recording player 16A may play a recording of a first song known by the at-risk person who has been identified by computer 20. Second recording player 16B may play a recording of a second song, and third recording player 16C may play a recording of a third song.

In some instances, a group of the recordings contained in data store 14 matches the ascertained identity of the at-risk person. Each one of the recordings in the group has a priority rank, as shown in Table I.

TABLE I

| Identity | Content | Priority Rank |
|---|---|---|
| John | Recording A | 1 |
| John | Recording B | 2 |
| John | Recording C | 3 |
| Mariko | Recording D | 1 |
| Mariko | Recording E | 2 |
| Mariko | Recording F | 3 |

Computer 20 is configured to select, according to the priority ranks, one of the recordings in the group to be the first recording to be played by first recording player 16C. For example, if the ascertained identity is John, computer 20 selects Recording A to be the first recording that is played by first recording player 16C and optionally by the second and third recording players. Alternatively, computer 20 selects Recordings A, B, and C to be played by the first, second, and third recording players, respectively, with staggered timing.

Computer 20 is configured to change the priority ranks according to an input, as shown in Table II.

TABLE II

| Identity | Content | Priority Rank |
|---|---|---|
| John | Recording A | 3 |
| John | Recording B | 2 |
| John | Recording C | 1 |
| Mariko | Recording D | 3 |
| Mariko | Recording E | 2 |
| Mariko | Recording F | 1 |

The input for changing the priority ranks can be based on a variety of factors. For example, the input can be any one of data on effectiveness of the first recording in attracting the person, environmental condition conditions, and the person's visitor history. For example, Recording C with priority rank of 3 for John in Table I may be a video recording of John's friend. After John's friend visits John at the caregiving facility, computer 20 may change the priority rank from 3 to 1, as shown in Table II. A record of the visit may be entered by a user into computer 20.

In another example, Recording D with priority rank of 1 for Mariko in Table I may be a first song. Over time, Mariko's response to the first song may decrease. The decrease may be inputted by a user into computer 20. Alternatively, the decrease may be detected by computer 20, such as during a prior incident when Mariko was previously identified at the controlled area, and the camera of identification sensor 12 showed that Mariko's travel was not delayed. As a result of any of these inputs, computer 20 may change the priority rank of Recording D from 1 to 3.

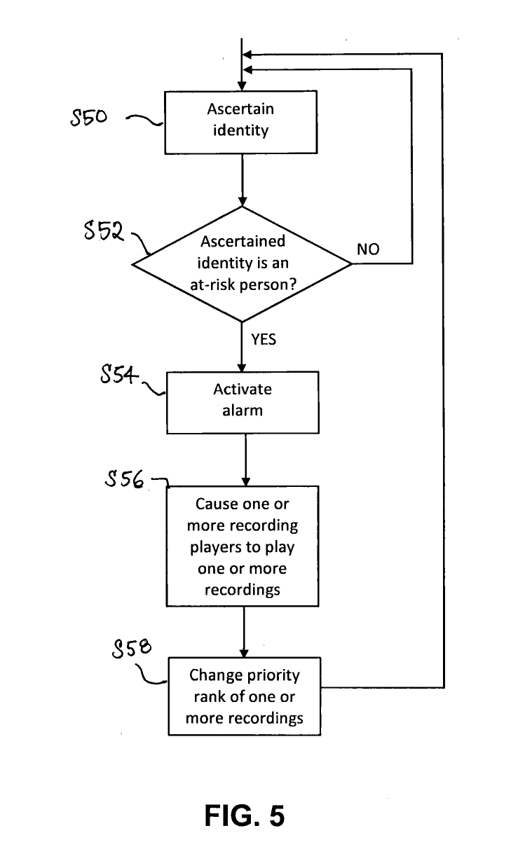

FIG. 5 illustrates an example entry-exit intervention method. At block S50, computer 20 ascertains an identity of a person according to sensor data 22, as previously described. The person is traveling toward or through a controlled area. Next at block S52, computer 20 determines whether the ascertained identity is an at risk person. For example, this may occur by determining that the ascertained identity matches any of a plurality of recordings 24 contained in data store 14. If NO at S52, alarm 18 is not activated, and none of the recording players is instructed to play a recording. If YES at S52, the process proceeds to blocks S54 and S56. At block S54, alarm 18 is activated.

At block S56, computer 20 causes one or more recording players to play one or more recordings 24 contained in data store 14, as previously described. The one or more recordings, when played, delays the at-risk person's travel. Meanwhile, the alarm summons another person to the at-risk person to prevent elopement. For example, block S56 may be limited to causing first recording player 16A to play a first recording among the plurality of recordings that matches the ascertained identity. In another example, block S56 may involve causing second recording player 16B to play the first recording with staggered timing relative to first recording player 16A, and may further involve causing third recording player 16C to play the first recording with staggered timing relative to second recording player 16B. Instead of selecting only a single recording, computer 20 may select first, second, and third recordings to be played by the first, second, third recording players, respectively with staggered timing.

Additionally or alternatively, the one or more players may facilitate a live discussion between the at-risk person and another person, such as a staff member of a caregiving facility. For example, the one or more players 16A, 16B, 16C may each have a microphone, and computer 20 may have a video camera and a microphone. The alarm alerts a first staff member situated by computer 20. Using the video camera and microphone, computer 20 transmits a video of a first staff member to each of the one or more players. At such time, the first staff member may coax or instruct the at-risk person to stay at the controlled area while a second staff member goes to the controlled area to intervene. With multiple players, the video of the first staff member may be played with staggered timing to lure the at-risk person away from the controlled area. Instead of a live discussion, the one or more players may play a video recording of a staff member that coaxes or instructs the at-risk person to stay or move away from the controlled area while a second staff member goes to the controlled area to intervene.

While several particular forms of the invention have been illustrated and described, it will also be apparent that various modifications may be made without departing from the scope of the invention. It is also contemplated that various combinations or subcombinations of the specific features and aspects of the disclosed embodiments may be combined with or substituted for one another in order to form varying modes of the invention. Accordingly, it is not intended that the invention be limited, except as by the appended claims.

1. A system for entry-exit intervention, the system comprising:
an identification sensor configured to output sensor data for identifying various persons;
a data store containing a plurality of recordings, each recording associated with one or more persons;
a first recording player comprising one or both of a speaker and a display screen;
an alarm; and
a computer, wherein the computer receives the sensor data, ascertains an identity of a person according to the received sensor data, determines whether the ascertained identity matches any of the recordings, and wherein when the ascertained identity matches any of the recordings, the computer activates the alarm and instructs the first recording player to play a first recording among the plurality of recordings that matches the ascertained identity.

2. The system of claim 1, wherein the identification sensor comprises a camera configured to output an image of the person as the sensor data, and the computer runs a facial recognition algorithm on the image to ascertain the identity of the person.

3. The system of claim 1, further comprising a second recording player comprising one or both of a speaker and a display screen, the second recording player being at least 2 meters from the first recording player, wherein
when the ascertained identity matches any of the recordings, the computer instructs the second recording player to play the first recording, and
the first and second recording players play the first recording with staggered timing.

4. The system of claim 1, wherein
the system further comprises a second recording player comprising one or both of a speaker and a display screen, the second recording player being at least 2 meters from the first recording player,
when the ascertained identity matches any of the recordings, the computer instructs the second recording player to play a second recording among the plurality of recordings that matches the ascertained identity, and
the first and second recording players play the recordings with staggered timing.

5. The system of claim 3, wherein the second recording player is from 2 meters to 20 meters from the first recording player.

6. The system of claim 1, further comprising a door adjacent to the first recording player, wherein the door is not locked when the first recording player plays the first recording.

7. The system of claim 1, wherein the first recording is one or any combination of a music recording, a song recording, and a video recording.

8. The system of claim 1, wherein
a group of the recordings contained in the data store matches the ascertained identity, each one of the recordings in the group has a priority rank,
the computer selects, according to the priority ranks, one of the recordings in the group to be the first recording to be played by the first recording player, and
the computer subsequently changes the priority ranks according to an input, the input being one of:
data on effectiveness of the first recording in attracting the person,
environmental condition conditions, and
the person's visitor history.

9. A method for entry-exit intervention, the method comprising:
ascertaining an identity of a person according to sensor data;
determining that the ascertained identity matches any of a plurality of recordings contained in a data store; and
upon such determination, activating an alarm and causing a first recording player to play a first recording among the plurality of recordings that matches the ascertained identity.

10. The method of claim 9, wherein the sensor data is an image of the person, and ascertaining the identity of the person comprises running a facial recognition algorithm on the image.

11. The method of claim 9, wherein
when the ascertained identity matches any of the recordings, causing a second recording player to play the first recording, the second recording player being at least 2 meters from the first recording player, and
the first and second recording players play the first recording with staggered timing.

12. The method of claim 9, wherein
when the ascertained identity matches any of the recordings, causing a second recording player to play a second recording among the plurality of recordings that matches the ascertained identity, the second recording player being at least 2 meters from the first recording player, and
the first and second recording players play the recordings with staggered timing.

13. The method of claim 11, wherein the second recording player is from 2 meters to 20 meters from the first recording player.

14. The method of claim 9, wherein a door is adjacent to the first recording player, and the door is not locked when the first recording player plays the first recording.

15. The method of claim 9, wherein the first recording is one or any combination of a music recording, a song recording, and a video recording.

16. The method of claim 9, wherein
determining that the ascertained identity matches any of the plurality of recordings contained in the data store comprises determining that the ascertained identity matches a group of the recordings,
each one of the recordings in the group has a priority rank,
causing the first recording player to play the first recording comprises selecting, according to the priority ranks, one of the recordings in the group to be the first recording to be played by the first recording player, and
the method further comprises changing the priority ranks according to an input, the input being one of:
data on effectiveness of the first recording in attracting the person,
environmental condition, and
the person's visitor history.

17. The method of claim 9, wherein
the person is traveling before the first recording is played by the first recording player,
the first recording, when played by the first recording player, delays the person's travel, and
the alarm summons another person to the person while the person's travel is delayed.

18. A non-transitory computer readable medium having stored thereon computer readable instructions that, when executed by a computer, cause the computer to perform a method for an entry-exit intervention, the method comprising:
ascertaining an identity of a person according to sensor data;
determining that the ascertained identity matches any of a plurality of recordings contained in a data store; and
upon such determination, activating an alarm and causing a first recording player to play a first recording among the plurality of recordings that matches the ascertained identity.

19. The non-transitory computer readable medium of claim 18, wherein the sensor data is an image of the person, and ascertaining the identity of the person comprises running a facial recognition algorithm on the image.

20. The non-transitory computer readable medium of claim 18, wherein
when the ascertained identity matches any of the recordings, causing a second recording player to play the first recording, the second recording player being at least 2 meters from the first recording player, and
the first and second recording players play the first recording with staggered timing.

21-24. (canceled)
